(19) United States
(12) Patent Application Publication
(10) Pub. No.: US 2007/0167897 A1
(43) Pub. Date: Jul. 19, 2007

(54) Apparatus for spinal positioning

(76) Inventor: Charles C. Schock, North Little Rock, AR (US)

Correspondence Address: WRIGHT, LINDSEY & JENNINGS LLP, 200 WEST CAPITOL AVENUE, SUITE 2300, LITTLE ROCK, AR, 72201-3699, US

(21) Appl. No.: 11/527,755

(22) Filed: Sep. 26, 2006

Related U.S. Application Data
(60) Provisional application No. 60/723,407, filed on Oct. 4, 2005.

(51) Int. Cl. A61F 5/00

(52) U.S. Cl. 602/32

(57) ABSTRACT

An apparatus for spinal positioning having a plurality of balloons that are selectively inflated and deflated by the user to customize the degree of the support and the location of the support to the user's back. In a preferred embodiment, the balloons number six and are arranged in two vertical rows so that the user can selectively provide support to one side of the back or the other to effect the best curve (lordosis) of the spine for maximum comfort. The balloons are incorporated in a jacket or brace worn about the torso of the user and fastened by an appropriate fastener. An air pump is operatively connected to the balloons by valving to direct air to or from each of the individual balloons. A control panel provides an array of switches to allow the user to select an individual balloon or balloons for inflation or deflation.

### CROSS-REFERENCE TO RELATED APPLICATIONS

This application claims the benefit of U.S. Provisional Application No. 60/723,407 filed Oct. 4, 2005, the disclosure of which is incorporated herein by reference.

### STATEMENT REGARDING FEDERALLY SPONSORED RESEARCH OR DEVELOPMENT

Not applicable.

### BACKGROUND OF THE INVENTION

The present invention relates to a device and method for positioning the spine of a user, and in particular, to such a device that employs a plurality of individually inflatable balloons disposed in a brace worn by the user.

The pain derived from a spinal injury may be relieved by positioning the spine to decompress the site of the injury.

It is known to use a compression garment for treating lymphedema and related illnesses. U.S. Pat. No. 6,852,089 discloses a compression garment that applies pressure at selected points in the human body. The garment includes a number of cells for holding pressurized air to selectively apply pressure to a particular area of the patient's body. The patent also discloses valving with manual controls to apply the pressurized air in measurable amounts to selected air cells or to deflate a cell after treatment. The garment may be fastened into position by means of Velcro or similar fasteners.

There are various patents that relate to relieving fatigue in drivers of automobiles using inflatable air cells to change the shape of a car seat. Some, such as U.S. Pat. No. 6,074,006 and Published Patent Application No. 2004/0174056 disclose air cells arranged on either side of the spine to provide support to the spine. Published Patent Application No. 2005/0127728 discloses an automobile seat with an array of individual air cells which are formed independently and connected to corresponding air compressors. Some of the air cells are arranged to either side of the spine of the user. U.S. Pat. No. 5,320,409 also shows a similar arrangement in which an automobile seat has an array of air cells connected by valving to an air compressor to allow the individual cells to be inflated or deflated as required. U.S. Pat. No. 4,570,676 discloses manual control of such air cells.

German Patent No. DE3334864 shows an automobile seat with an array of what are apparently air cells. Japanese Publication No. 04-005916 discloses an automobile seat with an array of what appears to be air cells individually connected to valving and a control mechanism. The cells are arrayed in a bilaterally symmetric fashion with four along each side of the spine in the seat back.

The particular problems associated with positioning the spine to relieve pain due to spinal injury are not addressed by the cited references. The cited references are not admitted to be prior art with respect to the present invention.

### BRIEF SUMMARY OF THE INVENTION

The present invention is a spine positioner for use in relieving the pain associated with spinal injuries by decompressing a site of annular weakness. The invention comprises a plurality of balloons that are selectively inflated and deflated by the user to customize the degree of the support and the location of the support to the user's back. In a preferred embodiment, the balloons number six and are arranged in two vertical rows so that the user can selectively provide support to one side of the back or the other to effect the best curve (lordosis) of the spine for maximum comfort. The balloons are incorporated into a jacket or brace worn about the torso of the user and fastened by a hook and loop type fastener or other suitable fasteners.

The present invention includes an air pump operatively connected by valving to direct air to or from each of the individual balloons. A control panel provides an array of switches to allow the user to select an individual balloon or balloons for inflation or deflation.

### BRIEF DESCRIPTION OF THE DRAWINGS

FIG. 1 is a posterior longitudinal view of the brace and balloons of the present invention shown in relation to the human spine.

FIG. 2 is a right side view of the brace of FIG. 1.

FIGS. 3A and 3B are a posterior longitudinal view and a right side view, respectively, of an L₄₅ intervertebral space showing an annulus fibrosus having a tear, bulge or similar weakness.

FIGS. 4A and 4B are views analogous to FIGS. 3A and 3B respectively. FIG. 4A shows the concave bending effect of a balloon inflated to the right side of the L₄₅ space thereby decompressing the site of the annular weakness. FIG. 4B shows the extension effect of a balloon inflated posterior to the annular weakness, thereby decompressing the annular weakness.

### DETAILED DESCRIPTION OF THE INVENTION

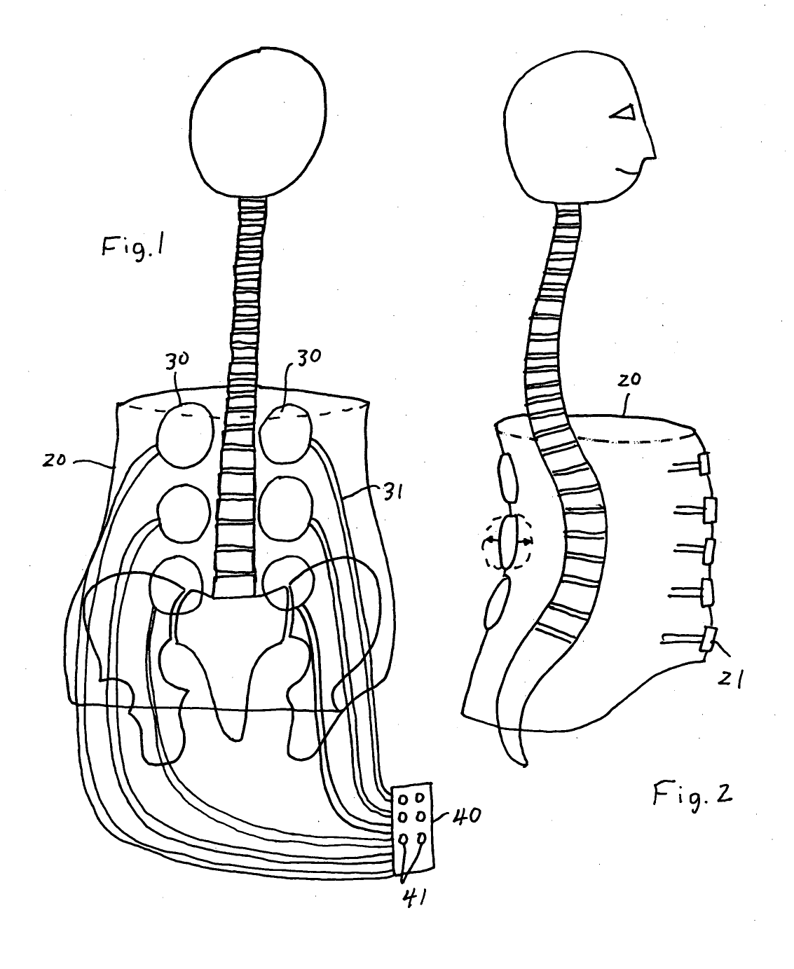

The preferred embodiments of the invention are described herein with reference to FIGS. 1-4B.

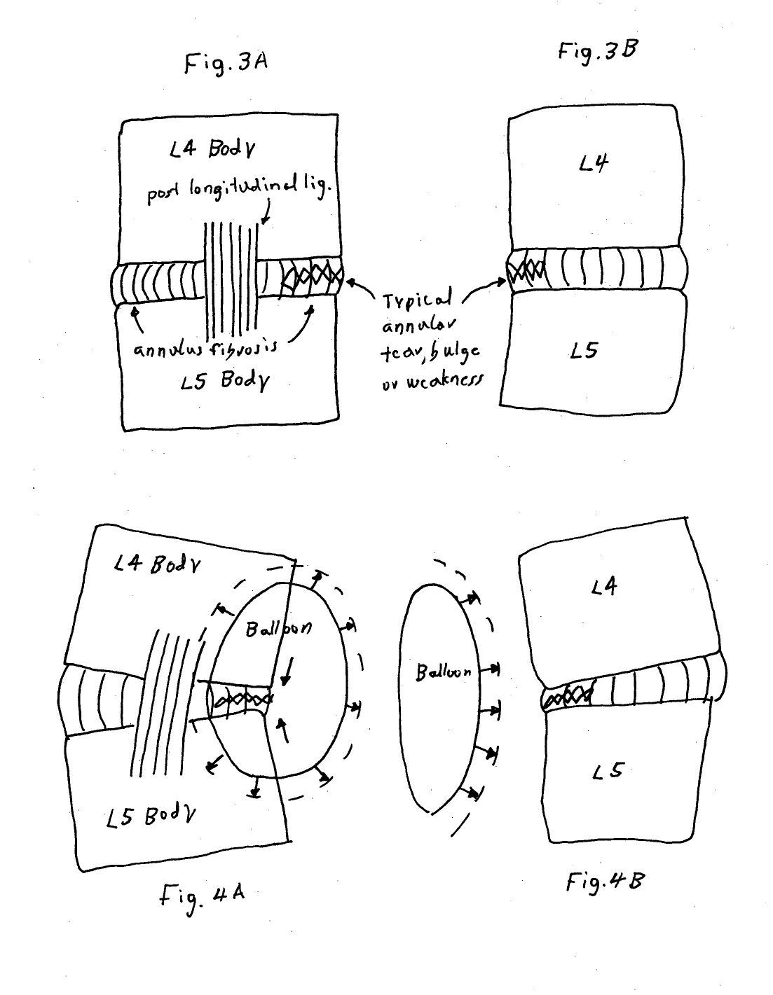

FIG. 3A is a posterior longitudinal view of an L₄₅ intervertebral space defined by the L₄ and L₅ vertebral bodies. A posterior longitudinal ligament is shown between the L₄ and L₅ vertebral bodies. Also, between the L₄ and L₅ vertebral bodies is an annulus fibrosus having a tear, bulge or similar weakness. FIG. 3B shows a right side view of the same.

The pain associated with the weakness of the annulus fibrosus may be relieved by decompressing the weakness in the annulus fibrosus as shown in FIGS. 4A and 4B. FIG. 4A shows the concave bending effect of a balloon inflated to the right side of the L₄₅ space thereby decompressing the site of the annular weakness. FIG. 4B shows the extension effect of a balloon inflated posterior to the annular weakness, thereby decompressing the annular weakness.

As shown in FIGS. 1 and 2, the present invention provides for a jacket or brace 20 that tightly encircles the lower torso. The brace 20 may be fastened by one or more fasteners 21. The fasteners may be of the hook and loop type of closure, or may be of any type of suitable fastener known to those in the art. The fastener or other means should enable the user to fit the brace 20 snugly about the torso.

The posterior portion of the brace 20 is provided with a plurality of balloons 30. The balloons 30 are preferably made of a tough leather-like material with an inner airtight rubber bladder. The balloons 30 are preferably about 4 to 5 inches (10 to 13 centimeters) in diameter and most preferably about 4 inches (10 centimeters) in diameter. The balloons 30 are connected by air tubing 31 to a pump (not shown). The pump may be of any suitable type known to those skilled in the art. The pump inflates the balloons 30 so that they expand toward the user as shown in FIG. 4B. While the balloons may also expand laterally, such is not required for the practice of the present invention.

The pump is operatively connected by valving (not shown) to direct air to or from each of the individual balloons 30. A control panel 40 provides an array of switches 41 to allow the user to select an individual balloon or balloons 30 for inflation or deflation. The switches 41 may operate in various modes in specific embodiments of the present invention. For example, activating a switch may allow a balloon to expand to a preset maximum size, while activating the switch a second time allows the balloon to fully deflate. As an alternative, activating the switch may allow the balloon to inflate or deflate in increments.

The user operates the invention to selectively inflate and deflate one or another of the balloons to customize the degree of the support and the location of the support to the user's back. In a preferred embodiment, the balloons number six and are arranged in two vertical rows so that the user can selectively provide support to one side of the back or the other to effect the best curve (lordosis) of the spine for maximum comfort and pain relief. The present invention is not limited to six balloons. Greater or lesser numbers may be employed as dictated by the needs of the specific user or the specific condition be treated.

The present invention has been described with reference to certain preferred and alternative embodiments that are intended to be exemplary only and not limiting to the full scope of the present invention as set forth in the appended claims.

What is claimed is:

1. An apparatus for positioning the spine of a user, comprising:
a jacket having means for fastening said jacket about the torso of the user;
a plurality of inflatable balloons disposed in said jacket along the spine of the user; and
an air pump operatively connected to said plurality of inflatable balloons by valving means for selectively inflating and deflating one or more of said plurality of balloons.

2. The apparatus of claim 1, wherein said plurality of inflatable balloons comprise two vertical rows of balloons, each of said rows disposed to a respective side of the spine of the user.

3. The apparatus of claim 2, wherein each of said vertical rows of balloons comprise three balloons.

4. The apparatus of claim 1, further comprising a control panel having an array of manual switches operatively connected to said valving means, each of said manual switches being associated with one of said plurality of inflatable balloons.

5. The apparatus of claim 1, wherein each of said plurality of inflatable balloons comprise a tough, leather-like outer layer and an air-tight inner bladder.

6. The apparatus of claim 1, wherein said means for fastening comprises hook and loop fastening material.

7. The apparatus of claim 1, wherein each of said plurality of balloons has a diameter between about 4 inches (10 centimeters) and about 5 inches (13 centimeters).

8. The apparatus of claim 7, wherein said diameter is about 4 inches (10 centimeters).

9. The apparatus of claim 4, further comprising means associated with each of said manuals switches for activating a switch and expanding an associated balloon to a maximum size and means associated with each of said manuals switches for activating a switch and deflating an associated balloon to a minimum size.

10. The apparatus of claim 4, further comprising means associated with each of said manual switches for inflating and deflating an associated balloon in increments.
